(19) United States
(12) Patent Application Publication
(10) Pub. No.: US 2026/0110603 A1
(43) Pub. Date: Apr. 23, 2026

(54) Conduit Systems with Internal Sensor Devices

(71) Applicant: ABB Schweiz AG, Baden (CH)

(72) Inventors: Rick Kukucka, Ann Arbor, MI (US); Anthony Cooper, Memphis, TN (US); James M. Baker, Southaven, MS (US)

(73) Assignee: ABB Schweiz AG, Baden (CH)

(21) Appl. No.: 18/918,852

(22) Filed: Oct. 17, 2024

(51) Int. Cl. G01N 1/22; G08B 21/18

(52) U.S. Cl. CPC G01N 1/2226; G08B 21/18

(57) ABSTRACT

A system for detecting contamination includes: a conduit component defining a sampling space; a sensor disposed inside the sampling space, the sensor sampling a fluid within the sampling space to detect, as a sampled value, a trace component in the fluid; and a processor disposed inside the conduit component, the processor receiving the sampled value, and transmitting the sampled value outside the conduit component as a contamination indication of the conduit component.

### FIELD

The present application generally relates to detecting contamination in a conduit assembly.

### BACKGROUND

Conduit assemblies are used for protecting electrical and/or communication wiring in harsh environments. To protect the wiring, the interiors of conduits in the conduit assembly should be sealed against gases or liquids that may damage the wiring, or may pose danger to equipment, operators, or products outside the conduit assembly.

There are some conduit assembly applications where it is important to know that the conduit assembly is sealed against moisture, and some applications where it is important to keep bacteria, algae, fungi, and other contaminants from growing inside the conduit assembly due to concern about contamination outside of the conduit. Such applications include facilitates in the food and beverage industry.

FIG. 1 shows a prior art conduit assembly 2. The conduit assembly 2 may include one or more sensor devices 4 associated with one or more conduit components 6, which conduit components 6 may include conduits 8, outlet boxes 10, and control panels 12, among others.

Currently, contamination of the conduit assembly 2 may only be detected by manually removing a cover of a conduit component 6 to perform a visual inspection. But if the contamination is not near the conduit component 6 that is visually inspected, the contamination may not be detected.

There is a need to address such problems to help identify a loss of sealing, to protect wiring within the conduit assembly, and to protect the environment in which the conduit assembly is disposed and/or products produced in that environment.

### SUMMARY

In an embodiment, the present invention provides a system for detecting contamination, the system comprising: a conduit component defining a sampling space; a sensor disposed inside the sampling space, the sensor being configured to sample a fluid within the sampling space to detect, as a sampled value, a trace component in the fluid; and a processor disposed inside the conduit component, the processor being configured to: receive the sampled value, and transmit the sampled value outside the conduit component as a contamination indication of the conduit component.

In an embodiment, the present invention provides a method of detecting contamination in a conduit component having a sampling space, the method comprising: sampling, using a sensor disposed inside the sampling space, a fluid within the sampling space to detect, as a sampled value, a trace component in the fluid; receiving, at a processor disposed inside the conduit component, the sampled value; and transmitting, by the processor, the sampled value outside the conduit component as a contamination indication of the conduit component.

### BRIEF DESCRIPTION OF THE DRAWINGS

FIG. 1 shows a prior art conduit assembly;

FIG. 2 shows systems for detecting contamination according to the present disclosure;

FIG. 3 shows various components of one system of FIG. 2;

FIG. 4 shows a conduit component having a sensor disposed therein;

FIG. 5 shows a detailed view of a conduit component; and

FIG. 6 shows a detailed view of a system for detecting contamination according to the present disclosure.

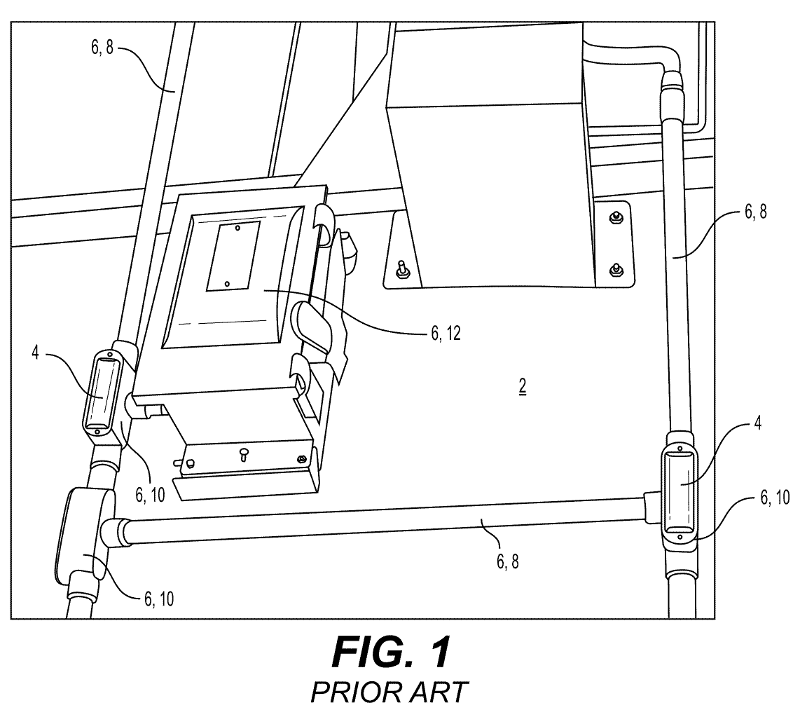

### DETAILED DESCRIPTION

In the context of a facility that processes food and/or beverages, a conduit assembly 2 should theoretically not be susceptible to contamination because 1) the conduit assembly 2 is generally closed-off and 2) the conduit assembly 2 itself is not typically an area that contains food and/or beverages, or their components. More specifically, the conduit assembly 2 is used to house electrical wiring or communications wiring for operating one or more machines in the facility, not for transporting products such as food and/or beverages.

Nevertheless, materials (e.g., food and/or beverages, or their components) somehow enter the conduit assemblies 2, which poses risk of contamination of the products produced by the facility, and ultimately sickness in the consumers of such products. Moreover, there are no known requirements for, or standards that require, cleaning out such conduit assemblies 2, and such cleaning is not performed in the ordinary course of business. Furthermore, current approaches for determining contamination of food and/or beverages measure the food and/or beverages directly as opposed to measuring a fluid (e.g., air) in a location where the food and/or beverages are not supposed to be.

To overcome the problems associated with the prior art, the present application describes systems and methods that, if there is contamination (e.g., bacteria, algae, fungi, etc.) within a conduit component, transmit a contamination indication outside of the conduit component without disassembling the conduit component.

One or more sensors may be disposed in the conduit component so as to facilitate the indication. The one or more sensors may detect one or more of temperature, moisture levels, gas concentration, or other information at specific points in the conduit assembly. Although the present application mostly discusses the use of a gas sensor, the discussion herein may be equally applicable to other types of sensors (e.g., temperature sensors, moisture sensors, etc.). The number of sensor devices in a conduit assembly may depend, for example, on the size of the conduit assembly and/or the application (i.e., measuring gas concentrations, temperatures, moisture levels, or other information), as well as the intended use of the sensed information.

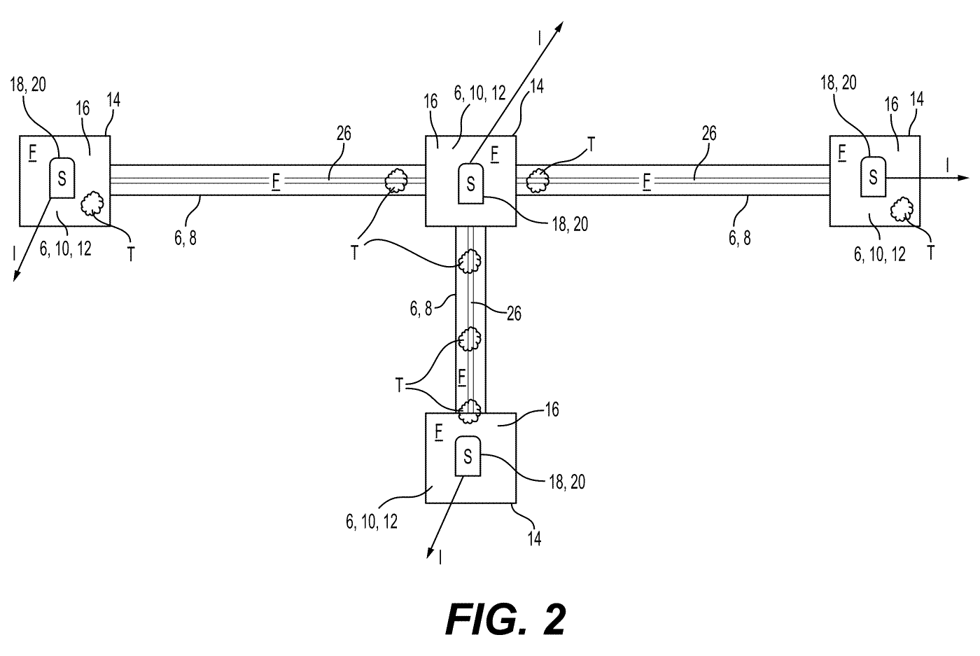

FIGS. 2-6 show various views and details of the systems and methods described herein. A system 14 for detecting contamination may include a conduit component 6 defining a sampling space 16. The conduit component 6 may be a conduit 8, an outlet box 10, and/or a control panel 12.

A sensor 18 may be disposed inside the sampling space 16. The sensor 18 may be configured to sample a fluid F within the sampling space 16 to detect, as a sampled value S, a trace component T in the fluid F.

System 14 may include a processor 20 disposed inside the conduit component 6. The processor 20 may be configured to receive the sampled value S (i.e., from sensor 18). Upon receipt of the sampled value S, the processor 20 may be configured to transmit the sampled value S outside the conduit component 6 as a contamination indication I of the conduit component 6.

In this manner, the systems and methods of the present application are able to notify a user, via contamination indication I, that a specific conduit component 6 is contaminated in some way (e.g., with bacteria, algae, fungi, etc.), all without needing to disassemble the conduit component 6. Upon receiving the contamination indication I (e.g., at a computer that may be either on the site of the conduit assembly 2 or located remotely therefrom), the user can then take remedial action to address the contamination (e.g., shut down associated machinery, sanitize, replace conduit components 6, etc.).

As shown in FIG. 2, a conduit assembly 2 could include multiple systems 14 connected by various conduits 8, each system 14 being associated with its own conduit component 6. Moreover, a system 14 could be disposed wholly within a conduit 8 so as to detect contamination with the conduit 8.

In an embodiment, the trace component T may be one or more of ethanol, ammonia, hydrogen sulfide, carbon dioxide, and carbon monoxide. These components are often produced by contamination (e.g., bacteria, algae, fungi, etc.), and may therefore be used to determine the presence of contamination in a conduit component 6.

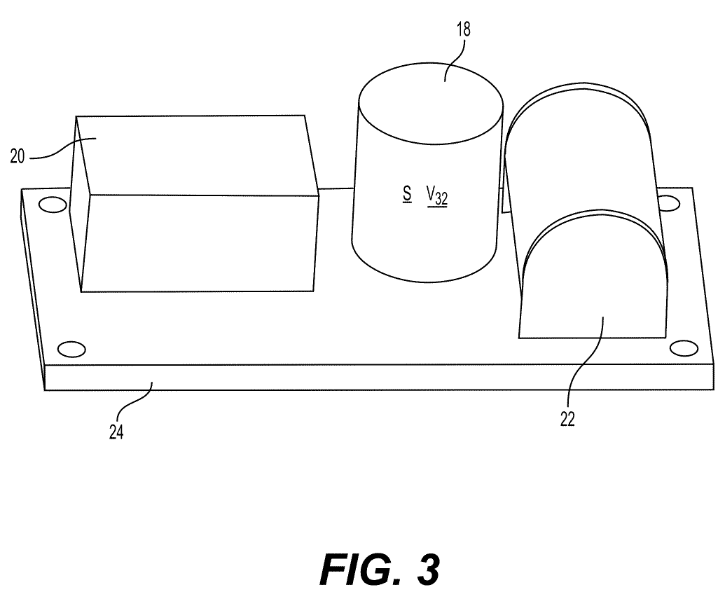

In an embodiment, a system 14 may also include an electrical power source 22, as shown in FIG. 3. The electrical power source 22 provides electrical power to one or more of the sensor 18 and the processor 20. The electrical power source 22 may be a battery disposed inside the conduit component 6. Alternatively, the electrical power source 22 may be an external power source disposed outside the conduit component 6 (e.g., an electrical wire providing electrical power to one or more of the sensor 18 and the processor 20).

In an embodiment, the processor 20 is configured to transmit the contamination indication I outside the conduit component 6 using one or more of Bluetooth, Wi-Fi, and cellular. Alternatively, the processor 20 may transmit the contamination indication I outside the conduit component 6 using a hard-wired connection to outside the conduit component 6.

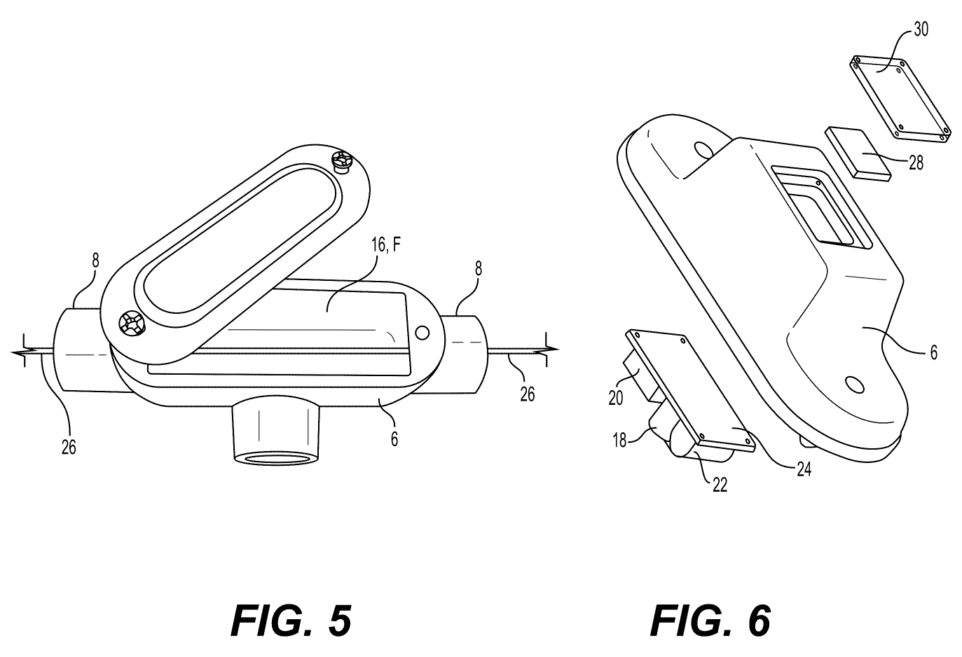

In an embodiment, at least one electrical wire 26 may be disposed inside the sampling space 16, as shown in FIG. 5. The at least one electrical wire 26 may provide electrical power or communication to one or more components associated with the conduit assembly 2, such as a food processing machine or a beverage processing machine. In this manner, the present application is directed to systems and methods for detecting contamination in an area where contamination is not typically expected to be (e.g., a sealed conduit carrying electrical wiring).

In an embodiment, the contamination indication I may be at least one of a visual alert and an audio alert. In this manner, the systems and methods described herein may be used to capture the attention of a user responsible for monitoring/controlling a machine or machines with which a conduit assembly 2 is associated, and/or someone in proximity of the conduit assembly 2.

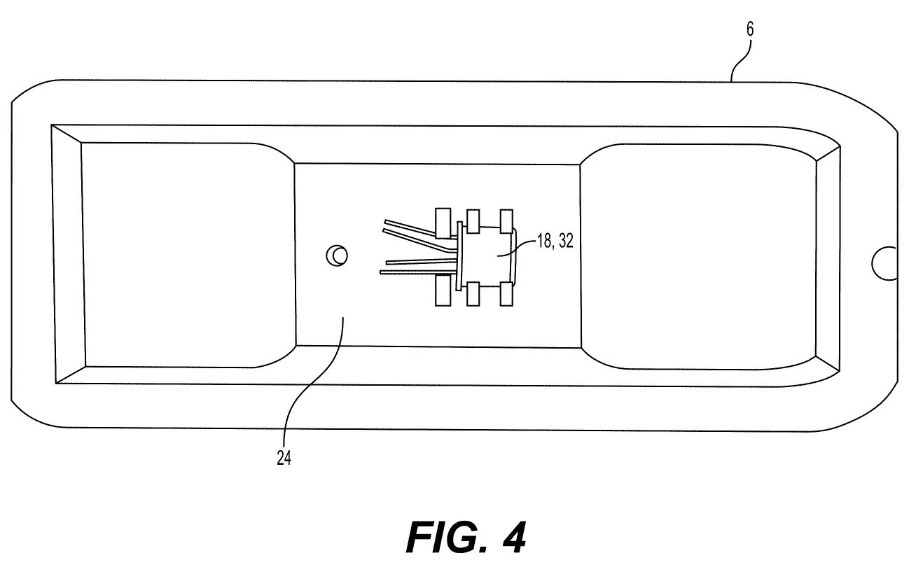

In an embodiment, if sensor 18 is a gas sensor, the sensor 18 may include a gas sensor resistor 32, as shown in FIG. 4. In this manner, the sampled value S could be a voltage drop V₃₂ across the gas sensor resistor 32. In this manner, presence of a trace component T in the fluid F in the sampling space 16 may result in the resistance of the gas sensor resistor 32 going down, which in turn results in voltage drop V₃₂ across the gas sensor resistor 32 increasing in magnitude, thus indicating contamination of the sample space with the trace component T.

In an embodiment, the system 14 includes a mount 24, such as a circuit board, that is disposed inside the conduit component 6, with the sensor 18 being attachable to the mount 24, as shown in FIGS. 3–4 and 6.

In an embodiment, the system 14 includes an antenna 28 disposed inside the conduit component to facilitate transmission of the contamination indication I outside the conduit component 6, as shown in FIG. 6. The system 14 may also include a window 30 on a portion of the conduit component 6 so as to facilitate transmission by the antenna 28 and/or processor 20.

A method of detecting contamination in a conduit component 6 having a sampling space 16 includes sampling, using a sensor 18 disposed inside the sampling space 16, a fluid F within the sampling space 16 to detect, as a sampled value S, a trace component T in the fluid F. The conduit component 6 may be a conduit 8, an outlet box 10, and/or a control panel 12.

The method may also include receiving, at a processor 20 disposed inside the conduit component 6, the sampled value S. The method may also include transmitting, by the processor 20, the sampled value S outside the conduit component 6 as a contamination indication I of the conduit component 6.

In an embodiment, the trace component T may be one or more of ethanol, ammonia, hydrogen sulfide, carbon dioxide, and carbon monoxide. These components are often produced by contamination (e.g., bacteria, algae, fungi, etc.), and may therefore be used to determine the presence of contamination in a conduit component 6.

In an embodiment, the processor 20 is configured to transmit the contamination indication I outside the conduit component 6 using one or more of Bluetooth, Wi-Fi, and cellular. Alternatively, the processor 20 may transmit the contamination indication I outside the conduit component 6 using a hard-wired connection to outside the conduit component 6.

In an embodiment, at least one electrical wire 26 may be disposed inside the sampling space 16, as shown in FIG. 5. The at least one electrical wire 26 may provide electrical power or communication to one or more components associated with the conduit assembly 2, such as a food processing machine or a beverage processing machine. In this manner, the present application is directed to systems and methods for detecting contamination in an area where contamination is not typically expected to be (e.g., a sealed conduit carrying electrical wiring).

In an embodiment, the contamination indication I may be at least one of a visual alert and an audio alert. In this manner, the systems and methods described herein may be used to capture the attention of a user responsible for monitoring/controlling a machine or machines with which a conduit assembly 2 is associated, and/or someone in proximity of the conduit assembly 2.

In an embodiment, a system 14 may also include an electrical power source 22, as shown in FIG. 3. The electrical power source 22 provides electrical power to one or more of the sensor 18 and the processor 20. The electrical power source 22 may be a battery disposed inside the conduit component 6. Alternatively, the electrical power source 22 may be an external power source disposed outside the conduit component 6 (e.g., an electrical wire providing electrical power to one or more of the sensor 18 and the processor 20).

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

### List of reference numerals

2 conduit assembly

4 sensor device

6 conduit component

8 conduit

10 outlet box

12 control panel

14 system

16 sampling space

18 gas sensor

20 processor

22 electrical power source

24 mount/circuit board

26 electrical wire

28 antenna

30 window

32 gas sensor resistor

F fluid

S sampled value

T trace component

I contamination indication

V₃₂ gas sensor resistor voltage drop

What is claimed is:

1. A system for detecting contamination, the system comprising:
a conduit component defining a sampling space;
a sensor disposed inside the sampling space, the sensor being configured to sample a fluid within the sampling space to detect, as a sampled value, a trace component in the fluid; and
a processor disposed inside the conduit component, the processor being configured to:
receive the sampled value, and
transmit the sampled value outside the conduit component as a contamination indication of the conduit component.

2. The system of claim 1, wherein the trace component comprises at least one of ethanol, ammonia, hydrogen sulfide, carbon dioxide, and carbon monoxide.

3. The system of claim 1, further comprising:
an electrical power source configured to provide electrical power to at least one of: the sensor and the processor.

4. The system of claim 3, wherein the electrical power source comprises a battery disposed inside the conduit component.

5. The system of claim 3, wherein the electrical power source comprises an external power source disposed outside the conduit component.

6. The system of claim 1, wherein the processor is configured to transmit the contamination indication using at least one of Bluetooth, Wi-Fi, and cellular.

7. The system of claim 1, wherein the processor is configured to transmit the contamination indication using a hard-wired connection to outside the conduit component.

8. The system of claim 1, further comprising:
at least one electrical wire disposed inside the sampling space.

9. The system of claim 1, wherein the contamination indication comprises at least one of a visual alert and an audio alert.

10. The system of claim 1, wherein the conduit component comprises at least one of a conduit, an outlet box, and a control panel.

11. The system of claim 1, wherein the sensor comprises a gas sensor resistor, and
wherein the sampled valued comprises a voltage drop across the gas sensor resistor.

12. The system of claim 1, further comprising:
a mount disposed inside the conduit component, the sensor being attachable to the mount.

13. The system of claim 1, further comprising:
an antenna disposed inside the conduit component.

14. A method of detecting contamination in a conduit component having a sampling space, the method comprising:
sampling, using a sensor disposed inside the sampling space, a fluid within the sampling space to detect, as a sampled value, a trace component in the fluid;
receiving, at a processor disposed inside the conduit component, the sampled value; and
transmitting, by the processor, the sampled value outside the conduit component as a contamination indication of the conduit component.

15. The method of claim 14, wherein the trace component comprises at least one of ethanol, ammonia, hydrogen sulfide, carbon dioxide, and carbon monoxide.

16. The method of claim 14, wherein the transmitting is via at least one of Bluetooth, Wi-Fi, and cellular.

17. The method of claim 14, wherein at least one electrical wire is disposed inside the sampling space.

18. The method of claim 14, wherein the contamination indication comprises at least one of a visual alert and an audio alert.

19. The method of claim 14, wherein the conduit component comprises at least one of a conduit, an outlet box, and a control panel.

20. The method of claim 14, wherein an electrical power source is disposed inside the sampling space, the electrical power source being configured to provide electrical power to at least one of the sensor and the processor.
